(19) United States
(12) Patent Application Publication
(10) Pub. No.: US 2008/0177054 A1
(43) Pub. Date: Jul. 24, 2008

(54) Automated Parallel Oligonucleotide Synthesis

(76) Inventor: David Evans, El Cajon, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/847,589

(22) Filed: Aug. 30, 2007

Related U.S. Application Data
(60) Provisional application No. 60/841,153, filed on Aug. 30, 2006.

(51) Int. Cl. C07H 1/00; B01J 19/00

(52) U.S. Cl. 536/25.31; 536/25.3; 422/116

(57) ABSTRACT

The invention is a system and method for the automated synthesis of biopolymers, such as polynucleotides. The synthesis reactions are conducted asynchronously in separate reaction volumes to minimize idle times for the machinery and to allow shorter polynucleotides to be removed when complete, rather than at the end of the entire run.

### CROSS-REFERENCE TO RELATED APPLICATIONS

This application claims the benefit of U.S. Provisional Application Ser. No. 60/841,153, filed 30 Aug. 2006, the entire contents of which is incorporated herein by reference in its entirety.

### FIELD OF THE PRESENT INVENTION

This invention relates generally to biopolymer synthesis and, more specifically, to devices and methods for the automated synthesis of high quality polynucleotides in parallel, allowing a single machine to produce a wider variety and increased number of polynucleotides concurrently with reduced synthesis times.

### BACKGROUND OF THE INVENTION

Many techniques in modern molecular biology employ synthetic polynucleotides, including the polymerase chain reaction (PCR), DNA sequencing, site directed mutagenesis, whole gene assembly, and single-nucleotide polymorphism (SNP) analysis. Unlike many other reagents used in molecular biology, polynucleotides are not generally available as stock items but are custom made to each user's specification. For example, the sequence, scale, purity, and modifications of a polynucleotide can be specified by the user.

Improvements in polynucleotide synthesis chemistry and processing technology have led to more rapid synthesis at a lower cost. However, polynucleotide synthesis remains a complex, multi-step process that requires a series of high efficiency chemical reactions.

Given the complex process, it is desirable to facilitate the production of a number of different polynucleotides concurrently during a single run of the synthesizer. Indeed, there has been a steady trend toward increasing the number of syntheses performed in parallel since the inception of automated DNA synthesis.

For example, U.S. Pat. No. 6,800,250 to Hunicke-Smith et al. discloses a synthesizer that can be used for the production of oligonucleotides using a movable synthesis block to expose the wells of a 96-well plate to injector nozzles. The MerMade-192 and MerMade-384 (BioAutomation, Plano, Tex.) DNA synthesizers similarly permit parallel synthesis of polynucleotides in 2 or 4 96-well plates, respectively. Further, U.S. Pat. No. 6,867,050 to Peck et al. also discloses apparatus and methods for parallel oligonucleotide synthesis of hundreds of different sequences and lengths at a time in a single 384-well plate. This reference also describes a system wherein four 384-well plates are employed. However, four injection heads are required and the reactions are duplicated on all four plates.

Thus, in all of the noted prior art methods and systems, the same reaction sequence is carried out simultaneously in each of the wells. Although different bases may be added to each well to form different polynucleotides, all of the polynucleotides are extended at the same rate and at the same time. This approach leads to significant inefficiencies. For example, even though the desired polynucleotide sequence may be completed in some of the wells, the automated run must continue while bases are added to the longer polynucleotides until the longest is complete. Further, certain reaction steps involve varied wait times for the reaction to complete. During these wait times, the system is idle.

Despite the improvements in automated synthesis, the reactions are time consuming. A significant limitation on the throughput of a synthesizer is simply the length of time required to produce a polynucleotide of a given length. For example, parallel synthesis of 20-mers can take 6 hours or longer. The inefficiencies noted above comprise a substantial portion of this time.

An alternate approach is disclosed in U.S. Pat. No. 6,258,323 to Hormann et al., which discloses an apparatus and method for multiple, simultaneous synthesis of compounds, including oligonucleotides. However, Hormann et al. require a stopper with multiple ports to be mounted to each vessel to allow the introduction of varying reagents. This approach rapidly becomes impractical, since the relatively complex injection system must be duplicated for each reaction vessel.

Thus, there exists a need for a device that allows for the rapid parallel synthesis of a number of high quality polynucleotides, for example, as raw materials suitable as building blocks for synthetic gene production. There is similarly a need for automated polynucleotide production that minimizes synthesis time while being highly flexible by allowing production of varied lengths of polynucleotides.

### SUMMARY OF THE INVENTION

In accordance with the above objects and those that will be mentioned and will become apparent below, the invention includes a method for the automated synthesis of polynucleotides comprising the steps of conducting a synthetic sequence comprising a repeating plurality of reactions in a first reaction volume, wherein each reaction is triggered by injecting reagents into the first reaction volume with an injection device and the reactions produce a polynucleotide; and conducting a synthetic sequence comprising a repeating plurality of reactions in a second reaction volume, wherein each reaction is triggered by injecting reagents into the second reaction volume with an injection device and the reactions produce a polynucleotide; wherein the synthetic sequence in the first reaction volume is asynchronous with the second synthetic sequence in the second reaction volume.

Preferably, the first reaction of the repeating plurality of reactions comprises adding a reagent to the first reaction volume and waiting a first period of time for the first reaction to occur, further comprising the steps of determining that sufficient time exists in the first period of time to conduct a second reaction in the second reaction volume; positioning the second reaction volume adjacent the injection device and injecting a reagent into the second reaction volume. In the noted embodiment, the first reaction volume is preferably located on a first plate and the second reaction volume is located on a second plate.

Also preferably, the method further includes the steps of completing a desired polynucleotide on the second plate, removing the second plate from a synthesis environment and continuing synthesis of another desired polynucleotide on the first plate. In this embodiment, the step of removing the second plate from a synthesis environment preferably comprises passing the second plate through an airlock while maintaining the synthesis environment.

In another embodiment of the invention, the step of removing the second plate from a synthesis environment comprises manipulating the second plate with at least one gloved access that maintains the synthesis environment.

According to the invention, the method can further comprise the step of conducting the synthetic sequence in a third reaction volume, wherein the plurality of reactions are triggered by injecting reagents into the third reaction volume with the injection device; wherein the synthetic sequence in the first reaction volume is asynchronous with the synthetic sequence in the third reaction volume. Preferably, the synthetic sequence in the second reaction volume is asynchronous with the synthetic sequence in the third reaction volume. In the noted embodiment, the method can further include conducting the synthetic sequence in a third reaction volume, wherein the plurality of reactions are triggered by injecting reagents into the third reaction volume with the injection device, determining that sufficient time exists in the first period of time to conduct a third reaction in the third reaction volume; positioning the third reaction volume adjacent to the injection device and injecting a reagent into the third reaction volume.

In an additional aspect of the invention, the method further comprises the step of conducting the synthetic sequence in a fourth reaction volume, wherein the plurality of reactions are triggered by injecting reagents into the fourth reaction volume with the injection device; wherein the synthetic sequence in the first reaction volume is asynchronous with the synthetic sequence in the fourth reaction volume. Preferably, the first, second, third and fourth reaction volumes are located on separate plates, such as 96-well plates.

In one embodiment of the invention, delivering a reagent includes injecting a reagent to an entire column of the reaction wells simultaneously.

In a preferred embodiment of the invention, the plurality of reactions comprise deblocking, coupling, capping and oxidizing.

According to the invention, polynucleotides having different sequences can be synthesized asynchronously. Further, polynucleotides having different lengths can be synthesized asynchronously.

The invention also includes a system for the automated synthesis of polynucleotides, comprising a first reaction volume and a second reaction volume, an injection device having injectors for delivering reagents into the first reaction volume and the second reaction volume; an xy table configured to movably position the first reaction volume and the second reaction volume adjacent the injection device, and a controller configured to conduct a repeating plurality of reactions corresponding to a synthetic sequence in the first reaction volume and the second reaction volume, wherein the synthetic sequence in the first reaction volume is asynchronous with the synthetic sequence in the second reaction volume.

Preferably, the first reaction of the repeating plurality of reactions comprises adding a reagent to the first reaction volume and waiting a first period of time for the first reaction to occur and wherein the controller is configured to determine that sufficient time exists in the first period of time to conduct a second reaction in the second reaction volume, operates the xy table so that the injection device is positioned adjacent the second reaction volume and injects a reagent into the second reaction volume.

Also preferably, the first reaction volume is located on a first plate and the second reaction volume is located on a second plate.

According to the invention, the system further comprises a dry box for maintaining a reduced moisture atmosphere surrounding the first reaction volume, the second reaction volume, the injection device and the xy table. Preferably, the dry box further comprises an air lock. Also preferably, the dry box further comprises at least one gloved access point.

In another embodiment of the invention, the system includes an integrated desiccator chamber configured to store one or more reagents. Preferably, the desiccator chamber and the dry box are configured so that one or more reagents are maintained under a reduced moisture atmosphere until delivery to the first and second reaction volumes.

In a further aspect of the invention, the system includes third and/or fourth reaction volumes, wherein the xy table is configured to movably position the third and/or fourth reaction volume adjacent the injection device and wherein the controller is configured to conduct the repeating plurality of reactions in the third and/or fourth reaction volume such that the synthetic sequence in the first reaction volume is asynchronous with the synthetic sequence in the third and/or fourth reaction volume. Preferably, the first, second, third and fourth reaction volumes are located on separate plates, such as 96-well plates.

According to the invention, the injection device can be configured to deliver reagents to an entire column of reaction wells simultaneously.

In one aspect of the invention, the controller includes software instructions comprising the steps of assessing the state of the first and second reaction volumes, determining the first reaction volume is waiting for a reaction to complete, determining the second reaction volume is ready for a subsequent reaction, and transmitting commands that cause the injection device to deliver to the second reaction volume to initiate the subsequent reaction.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is an overall view, showing an apparatus for the automated synthesis of polynucleotides, according to the invention;

FIG. 2 shows a schematic view of the injection head and xy table of a synthesizer embodying features of the invention;

FIG. 3 shows a detailed schematic of a 96-pin injection head, embodying features of the invention;

FIG. 4 shows a schematic view of reagent containers and valves of an automated polynucleotide synthesizer, according to the invention;

FIG. 5 shows a schematic view of the control hardware of an automated polynucleotide synthesizer, according to the invention;

FIG. 6 shows a diagram of a gas supply system for an automated polynucleotide synthesizer, according to the invention;

FIG. 7 shows a diagram of a reagent container pressure system for an automated polynucleotide synthesizer, according to the invention;

FIG. 8 shows a diagram of a wash system for an automated polynucleotide synthesizer, according to the invention;

FIG. 9 shows a diagram of a vacuum waste system for an automated polynucleotide synthesizer, according to the invention; and

FIG. 10 shows a flowchart of steps performed in determining the sequence of reaction steps in a plurality of reaction volumes, according to the invention.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise.

This invention provides a method for the automated synthesis of polynucleotides comprising the steps of conducting a synthetic sequence comprising a repeating plurality of reactions in a first reaction volume, wherein each reaction is triggered by injecting reagents into the first reaction volume with an injection device and the reactions produce a polynucleotide; and conducting a synthetic sequence comprising a repeating plurality of reactions in a second reaction volume, wherein each reaction is triggered by injecting reagents into the second reaction volume with an injection device and the reactions produce a polynucleotide; wherein the synthetic sequence in the first reaction volume is asynchronous with the second synthetic sequence in the second reaction volume.

In one embodiment, the first reaction of the repeating plurality of reactions comprises adding a reagent to the first reaction volume and waiting a first period of time for the first reaction to occur. Preferably, the method further comprises the steps of: determining that sufficient time exists in the first period of time to conduct a second reaction in the second reaction volume, moving the injection device to the second reaction volume and injecting a reagent into the second reaction volume.

The concepts of the invention can be extended to three or more reaction volumes, such that the synthesis reactions are conducted asynchronously in each of the reaction volumes. Preferably, each of the three or more reaction volumes are located on separate plates. Additional reaction volumes can be located on each plate, wherein synthesis reactions on each plate occur synchronously.

As used herein, the terms “polynucleotide” and “oligonucleotide” are intended to mean two or more nucleotides linked together through a covalent bond and are used interchangeably. For example, nucleotides can be linked together through a phosphodiester bond. A polynucleotide can contain the four nucleotides adenine, guanine, cytosine, and thymine or nucleotide analogues and derivatives such as inosine, dideoxynucleotides or thiol derivatives of nucleotides. Different chemical forms of nucleotides such as nucleosides or phosphoramidites can be used to generate a polynucleotide. In addition, nucleotides can further incorporate a detectable moiety such as a radiolabel, a fluorochrome, a ferromagnetic substance, a luminescent tag or a detectable moiety such as biotin. Polynucleotides also include, for example, RNA and peptide nucleic acids (PNAs).

As used herein, “reagent” is intended to mean a substance used in a chemical reaction to detect, examine, measure, or produce other substances. When a reagent is used in the production of a desired substance, such as a polynucleotide, the reagent can be used at any stage in the production of the desired substance. For example, a reagent can be a precursor such as a nucleotide-solution which is used at the beginning of the production of a polynucleotide. In addition, a reagent can be a solution used later in the production of a polynucleotide such as a wash solution that is used to wash away un-bound nucleotides. For example, an acetonitrile wash solution is a reagent that can be used in the production of polynucleotides. Reagents include, for example, amidites, deblock, oxidizer, activator, capping reagents, and acetonitrile wash solution.

As used herein, the term “synthesis platform” of an automated polynucleotide synthesizer is intended to mean the surface of an automated polynucleotide synthesizer that contains or can hold a reaction vessel or chamber, or vessels or chambers where the polynucleotide synthesis occurs. For example, the synthesis platform can contain one or more wells or columns or plates where the polynucleotide synthesis reaction can occur. Several automated polynucleotide synthesizers are commercially available. For example, the Applied Biosystems ABI 381A and Perseptive Biosystems 8905 are standard polynucleotide synthesizers that are commercially available. Also, for example, a polynucleotide synthesizer can be a custom made synthesizer such as the MerMade polynucleotide synthesizer (see Rayner et al., Genome Research 8:741-747 (1998), which is incorporated herein by reference). Specific details regarding automated polynucleotide synthesis under anhydrous conditions are disclosed in U.S. Patent Application Publication No. 20040223885A1, published Nov. 11, 2004, which is hereby incorporated by reference in its entirety.

Methods for synthesizing polynucleotides (also known as oligonucleotides) are known in the art and can be found described in, for example, Oligonucleotide Synthesis: A Practical Approach, Gate, ed., IRL Press, Oxford (1984); Weiler et al., Anal. Biochem. 243:218 (1996); Maskos et al., Nucleic Acids Res. 20(7):1679 (1992); Atkinson et al., Solid-Phase Synthesis of Oligodeoxyribonucleotides by the Phosphitetriester Method, in Oligonucleotide Synthesis 35 (M. J. Gait ed., 1984); Blackburn and Gait (eds.), Nucleic Acids in Chemistry and Biology, Second Edition, New York: Oxford University Press (1996), and in Ansubel et al., Current Protocols in Molecular Biology, John Wiley and Sons, Baltimore, Md. (1999).

Solid-phase synthesis methods for generating arrays of polynucleotides and other polymer sequences can be found described in, for example, Pirrung et al., U.S. Pat. No. 5,143,854 (see also PCT Application No. WO 90/15070), Fodor et al., PCT Application No. WO 92/10092; Fodor et al., Science (1991) 251:767-777, and Winkler et al., U.S. Pat. No. 6,136,269; Southern et al. PCT Application No. WO 89/10977, and Blanchard PCT Application No. WO 98/41531. Such methods include synthesis and printing of arrays using micropins, photolithography and ink jet synthesis of oligonucleotide arrays.

Methods for synthesizing large nucleic acid polymers by sequential annealing of polynucleotides can be found described in, for example, in PCT application No. WO 99/14318 to Evans and U.S. Pat. No. 6,521,427 to Evans. All of the above references are incorporated herein by reference in their entirety.

A typical polynucleotide synthetic sequence comprises the core reaction steps of deprotection of an immobilized dimethoxytrityl (DMT) protected nucleoside or polynucleotide, chain extension by coupling an activated and appropriately protected nucleotide monomer derivative to the immobilized nucleoside or polynucleotide, capping to chemically inactivate immobilized nucleoside or nucleotide chains that failed to react with a nucleotide monomer during the extension reaction, and oxidation to form a nucleotide chain comprising a pentavalent phosphate triester. These core steps are repeated as part of the “reaction cycle” necessary to synthesize a given polynucleotide. Each reaction cycle adds a single nucleotide to an immobilized nucleoside or polynucleotide and creates a larger, immobilized polynucleotide. The final step at the end of a given polynucleotide synthesis is a cleavage reaction that releases the newly synthesized polynucleotide chain from the substrate it has been immobilized on.

As used herein, the term “synchronous” means conducting at least two polynucleotide synthetic sequences in separate reaction volumes such that each individual reaction cycle in the synthetic sequence is initiated at the same time in each reaction volume in which polynucleotide chain extension is necessary.

In a synchronous synthetic sequence involving at least two polynucleotides, each necessary extension reaction is initiated in each separate reaction volume at the same time and all other synthesis cycle reactions are also performed at the same time. The result is that in a synchronous synthetic sequence each necessary synthesis cycle performed in separate reaction volumes starts and ends at the same time.

In some instances at least two of the polynucleotides to be synthesized differ from each other in size, such that one polynucleotide comprises a different number of nucleotides and is larger than another polynucleotide molecule to be synthesized. More reaction cycles are necessary to synthesize the larger polynucleotide and fewer are needed to synthesize the smaller polynucleotide. Thus, a longer time period is required to make the larger polynucleotide and a shorter time period is required to make the smaller polynucleotide.

In a synchronous synthetic sequence no new reaction cycles can be initiated until slower syntheses or slower synthesis cycle reactions are completed in all other separate reaction volumes. This creates considerable inefficiency, because the time required to complete a synchronous synthetic sequence can only be as fast as the slowest, most rate-limiting individual polynucleotide synthesis or synthesis cycle reaction to be performed.

As used herein, the term “asynchronous” means at least two polynucleotide synthetic sequences conducted in separate reaction volumes such that each individual reaction cycle in the synthetic sequence is initiated at different times in each reaction volume in which polynucleotide chain extension is necessary.

In an asynchronous synthetic sequence involving at least two polynucleotides each necessary extension reaction can be initiated in each separate reaction volume at different times. One result is that synthesis cycle reactions in each separate reaction volume may be performed at different times in an asynchronous synthetic sequence. This means that syntheses in separate reaction volumes can proceed toward completion independently of slower synthetic processes, such as syntheses of larger polynucleotides or slower synthesis cycle reactions, occurring in other separate reaction volumes. The result in an asynchronous synthetic sequence is that no time is lost waiting for the slowest, most rate-limiting individual polynucleotide synthesis or slower synthesis cycle reactions occurring in other separate reaction volumes to be completed.

Polynucleotides can be generated on commercial nucleic acid synthesizers using phosphoramidite chemistry. The Practical Approach series has reviewed phosphoramidite and alternative synthetic strategies (Brown, T., and Dorkas, J. S. Oligonucleotides and Analogues a Practical approach, Ed. F. Eckstien, IRL Press Oxford UK (1995)).

Chemical synthesis of polynucleotides is a process in which four building blocks (base phosphoramidites) are connected as a linear polymer. In addition to the component bases, a number of reagents are required to assist in the formation of internucleotide bonds, oxidize, cap, detritylate, and deprotect. Automated synthesis can be performed on a solid support matrix that serves as a scaffold for the sequential chemical reactions; a series of valves and timers to deliver the reagents to the matrix, and finally a post-synthesis processing stream that can include purification, quantification, product quality control, and lyophilization.

Some of the standard DNA bases (Guanine (G), Cytosine (C), and Adenine (A)) contain primary amines that are reactive; therefore, the primary exocyclic amines can be modified with protecting groups so as to not participate in unwanted reactions during synthesis. Further, the four phosphoramidites contain a phosphorus linkage that similarly needs to be protected. Chemical groups used to protect these sensitive sites can remain intact during the DNA synthesis cycle yet can be readily removed after synthesis so that normal, unmodified DNA results. A number of different protecting strategies have been developed. For example, phosphoramidites with p-cyanoethyl protected phosphorus can be used. For the heterocyclic bases, protection of primary amines is often provided by a benzoyl group for adenine and cytosine and either a dimethylformamidine or isobutyl group for guanine. Thymine (T), which lacks a primary amine, does not require base protection. These protecting groups are stable under conditions used during synthesis, but are rapidly and effectively removed by treatment with ammonia.

It is also desirable to block the 5′-OH of the base-phosphoramidites so that activated monomers do not react with themselves but can only react with the 5′-OH on the growing polynucleotide chain tethered to the solid support. Current chemistry, for example, employs a dimethoxytrityl (DMT) group. After condensation, the DMT group is cleaved from the newly added DNA base by treatment with acid. The released DMT cation is orange and therefore, the progress of the DNA coupling efficiency can be monitored by analyzing the spectrophotometric reading at 490 nm.

The 3′ hydroxyl group of the deoxyribose sugar is derivatized with a highly reactive phosphitylating agent. The phosphate oxygen on this group is usually masked by the β-cyanoethyl moiety that can be removed by β-elimination using ammonia hydroxide treatment at elevated temperatures.

Thus, the phosphoramidite polynucleotide synthesis cycle comprises the repeating steps: Deblocking; Activation/coupling; Capping; and Oxidation.

Automated synthesis can be done on solid supports, usually controlled pore glass (CPG) or polystyrene. CPG is loaded into a small column that serves as the reaction chamber. A loaded column is attached to reagent delivery lines on a DNA synthesizer and the chemical reactions proceed under computer control. Bases are added to the growing chain in a 3′ to 5′ direction (opposite to enzymatic synthesis by DNA polymerases). Although “universal” supports exist, synthesis is more often begun using CPG that is already derivatized with the first base, which is attached via an ester linkage at the 3′-hydroxyl. Synthesis starts with the first base attached to the CPG solid support and elongates in a 3′ to 5′ direction. CPG particles are relatively large and are porous, containing channels that greatly increase the surface to volume ratio, allowing the reaction to be done in a small reaction chamber using small volumes of reagents. The CPG is positioned in a “column” between two filter frits; with a reagent entry port on one end and an exit port on the other.

During synthesis, both full-length polynucleotides and truncation products or partial polynucleotide products remain attached to the CPG support. Following synthesis, the species are similarly cleaved and recovered so that the final reaction product is a heterogeneous mixture of wanted and unwanted species. Impurities accumulate to a greater degree as polynucleotide length increases. Furthermore, cleaved protecting groups are also present. At this point, polynucleotides are traditionally “desalted”, a process in which small molecule impurities (protecting groups and short truncation products) are removed using gel filtration or organic solid-phase extraction (SPE) methods to complete the post-synthesis handling.

Use of desalted polynucleotides with no additional purification can be appropriate when using short primers in simple applications, such as routine PCR or DNA sequencing. However, n−1 and other truncation or partial polynucleotide species can lead to deletion mutants if used in cloning, site-directed mutagenesis or gene assembly applications. Purification by PAGE or HPLC can be used to remove truncated or partial polynucleotide species.

The invention provides an apparatus for performing asynchronous parallel synthesis in a plurality of reaction volumes. Subsequent synthesis reactions are conducted in other reaction volumes during the wait time of a reaction occurring in a given reaction volume.

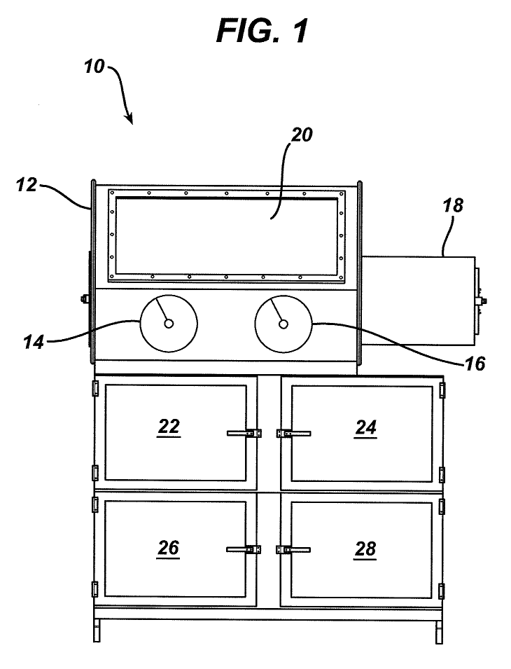

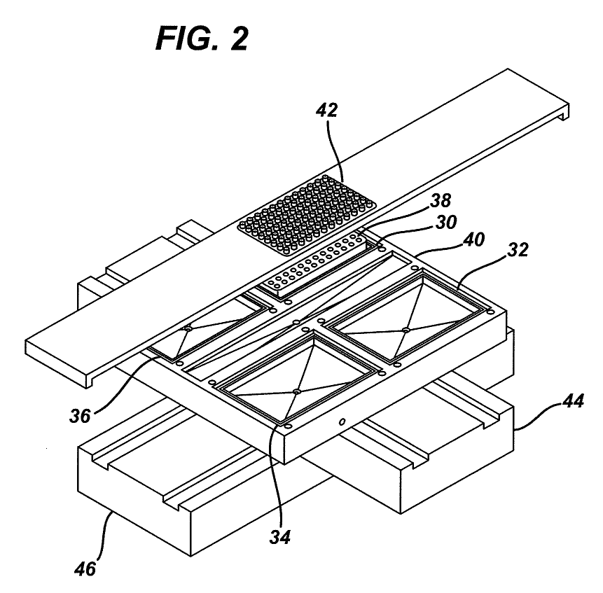

An example of an apparatus of the invention is shown in FIG. 1 with various views and close-up diagrams of an apparatus shown in FIGS. 2-9.

As shown in FIG. 1, one embodiment of the present invention includes a cabinet 10 configured to house the various components of the automated oligonucleotide synthesizer, such as the synthesis plates, the xy table, the injector head, the reagents, the controller and the plumbing, all of which are described in detail below. By enclosing all the components of the synthesizer within a single cabinet, a closed continuous system is provided to improve the efficiency of the synthesis reactions. Generally, cabinet 10 includes a dry box 12 comprising a controlled environment hood over the synthesis platform. Gloved access points 14 and 16 allow the operator to manipulate plates and machinery within dry box 12 without contaminating the atmosphere with moisture. Dry box 12 also includes air lock 18 to allow plates and other materials to be introduced or removed from dry box 12 while maintaining a controlled atmosphere. For example, this allows one plate to be removed from the dry box during a run, even if synthesis continues on the other plates. One or more viewing windows 20 allow the operator to monitor the synthesis reactions and facilitate any operations involving the gloved access points and air lock.

As one having ordinary skill in the art will appreciate, the dry box 12, gloved access points 14 and 16 and air lock 18 can be constructed from any suitable group of natural and synthetic materials, for example, a moisture- and solvent-resistant material such as Pyrex glass, stainless steel, polypropylene, rubber, latex or Teflon can be used. In one embodiment, the dry box is made of a moisture-resistant material and sealed over a synthesis platform so as to provide a closed continuous anhydrous system for oligonucleotide synthesis. As used herein, the term “moisture-resistant” is intended to mean a substance that is impermeable to water vapor and liquid.

As used herein, the term “closed continuous anhydrous system” is intended to mean a system that can monitor and react to the amount of moisture in the system in such a way that maintains homeostasis. The desired homeostatic state can be set by the user in terms of the percent of moisture or humidity in the system. For example, a closed continuous anhydrous system can be an enclosed area where the amount of moisture is constantly monitored and adjusted to exclude as much moisture as possible from the system. In addition, for example, other variables can be regulated in a closed continuous anhydrous system such as pressure levels.

The term “anhydrous” is intended to mean a low water content. Water content can be measured in several ways, for example, as percent of humidity using a humidity meter. An anhydrous system can have a low level of humidity or moisture. For example, an anhydrous system can have 5% relative humidity (RH) or less, 4% relative humidity or less, 3% relative humidity or less, 2% relative humidity or less, 1% relative humidity or less, 0.5% relative humidity or less, or no detectable relative humidity. Water content can also be measured in parts per million (ppm) units. For example, the water content in an organic solvent can be 10 ppm or less for anhydrous organic solvents.

Cabinet 10 also includes doors 22 and 24 to provide access to an integrated desiccator chamber within cabinet 10. The desiccator chamber maintains the synthesis reagents under a constant flow of dry nitrogen during operation. Accordingly, the reagents are kept in a continually dry environment during system operation to ensure the synthesis reactions are carried out in anhydrous conditions. Further, the desiccator chamber provides secondary spill containment and fire protection.

As also shown in FIG. 1, cabinet 10 has doors 26 and 28 to provide access to the control hardware, including a computer, and valves, tubing, waste containers and other plumbing apparatus of the synthesizer, all described in more detail below.

In one embodiment, the polynucleotide synthesizer of the invention is configured to synthesize polynucleotides in a single run using standard phosphoramidite chemistry in four standard 96-well plates. The machine is capable of making a combination of standard, degenerate, or modified polynucleotides in each plate. The run time can be about 17 hr or less for four plates of 20-mers and a reaction scale of 40 nM. The reaction vessel can be a standard polypropylene 96-well plate with a hole drilled in the bottom of each well.

As shown schematically in FIG. 2, the synthesis platform of this embodiment comprises four separate vacuum chucks 30, 32, 34 and 36, which are configured to receive plates 38, such as a 96-well plate (only one shown for clarity). Chucks 30-36 are mounted on an xy table 40, which is configured to position each plate at a desired position under injection head 42. As can be appreciated, each well of each plate must be accurately positioned under the appropriate reagent injection line of injection head 40 to allow the reagent to be injected into the desired well.

Xy table 40 generally comprises a track 44 capable of translocating vacuum chucks 30-36 in one direction and a track 46 capable of translocating the chucks in a perpendicular direction. In one embodiment of the invention, tracks 44 and 46 comprise a linear motor positioner, such as a Daedal linear motor (Parker Hannifin Corp., Irwin, Pa.). In this embodiment, a magnetic encoder to provides precise control over table position and allows the table to be manually moved without affecting the calibration. Other suitable mechanisms for xy tables as known in the art can also be employed.

As shown in FIG. 2, injection head 42 is mounted over xy table 40, so that the plates are moved underneath the chemical injection head to the proper position. The combination of the two chambers is designed to exclude contaminants from the reactions.

The four synthesis plates can be individually mounted inside vacuum chucks 30-36 to allow drainage of the reagent chemicals after each stage. The vacuum chuck consists of two parts that bolt together around the plate. The lower half of the chuck contains a gasket to provide a seal between the plate and the chuck, and a drain line that is connected to a vacuum. The plates can be mounted in the vacuum chucks through air lock 18 using gloved access points 14 and 16.

The chucks used to mount the synthesis plate can be modified to have a deeper collection basin than in a standard synthesizer. For example, the chucks can be modified to have an 8 mm deeper collection basin. This modification is useful so that if reagents leak through the filter plate during a reaction step, the reagent will not fill the basin and cross contaminate different synthesis microwells in the filter synthesis plate.

In alternate embodiments of the invention, the plates can be fixed while the injection head is moved to position it at the proper location relative to the plates.

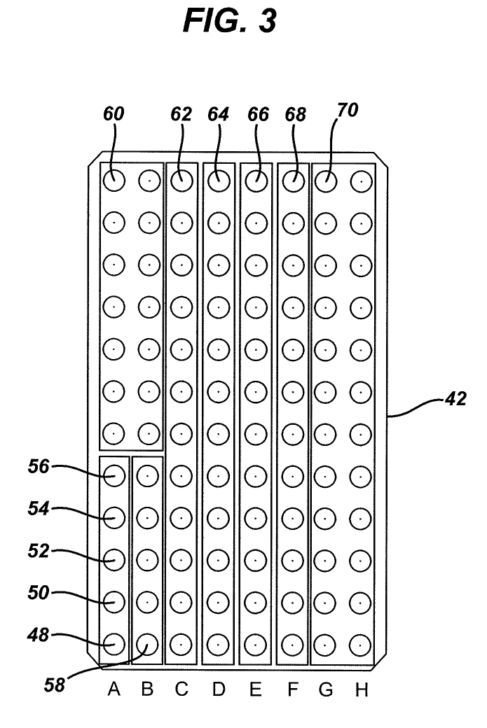

In this embodiment of the invention, the reagents are introduced into the wells of plates 38, via the chemical injection head 42. FIG. 3 shows the injector pin layout of injection head 42. As shown, injection head 42 comprises 8 rows of 12 injector pins. In row A, injector pins 48, 50, 52 and 54 are configured to deliver the phosphoramidites A, T, C and G. Injector pin 56 is reserved in this configuration. In row B, injector pin group 58 is configured to deliver the appropriate activator reagent in conjunction with the phosphoramidites delivered by injector pins 48-54. By providing a separate activator injector pin for each phosphoramidite, movement of table 40 is minimized, and the time required to deliver the bases to the entire plate is reduced. Pin group 60 is reserved, and can be used to deliver modified bases in conjunction with an activator or other reagents. In row C, pin group 62 provides a full bank of pins for delivering the deblock reagent to an entire column of wells simultaneously. Similarly, in rows D and E, pin groups 64 and 66 are configured to deliver coupling reagents A and B simultaneously to a full column of 12 wells. Row F comprises pin group 68, which is configured to deliver the oxidizer reagent to a full column of wells. Finally, rows G and H comprise pin group 70, which are configured to deliver a double volume of wash to a full column of wells.

As referenced above, the reagents can be stored in bottles in the desiccator chamber and delivered via Teflon tubing to injection head 42. Lead-throughs can be used to bring the tubing from the desiccator chamber to dry box 12. Silicone sealant can be used to produce a seal through which the tubing enters the lead-through. DC solenoid valves can be used to regulate the flow of reagents into the wells, which in turn can be controlled individually by solid-state relays that are switched by the software described below. The valves can be controlled by a National Instruments NB-DIO-96 card. Signals are sent from the card to three banks of relay cards (each card contains eight relays). Two cards control the DC valves for reagent injection; the third card controls the AC valves that are used for argon and vacuum systems. The AC and DC voltage sources for the motors and valves are provided by the voltage supply box. The smallest injection volumes obtainable with these valves is <20 μl. However, enhanced mixing of the reagents in the wells can be achieved with injection volumes in the range of at least approximately 50 μl.

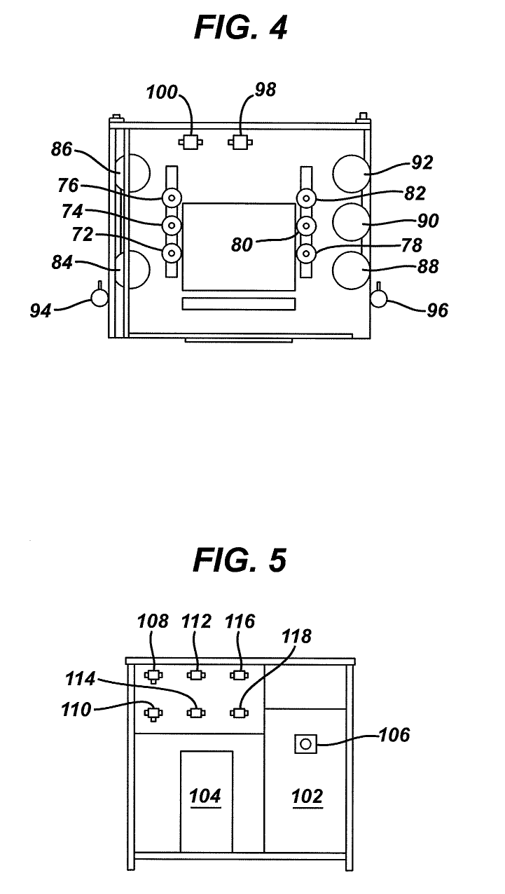

FIG. 4 schematically shows the reagents and associated plumbing contained within the desiccator chamber of cabinet 10. Generally, the synthesis reagents can be stored inside containers, such as containers 72, 74, 76 and 78 used to hold nucleotide base reagents adenine, thymine, cytosine, and guanine. Container 80 is used to hold the final phosphate phosphoramidite base. Additional containers (not shown) can be used for additional nucleotide base reagents, such as modified bases. Further, container 82 holds deblock reagent, container 84 holds oxidizer reagent, container 86 holds activator reagent, containers 88 and 90 hold capping reagents and container 92 holds a wash solution such as acetonitrile.

Tubing (not shown) connects the reagent containers to injection head 42 in the dry box 12 through individual solenoid valves. In one embodiment, an apparatus of the invention contains a flow through gas dryers 94 and 96 connected to tubing that connects the reagent containers 72-92 to a gas supply. Also, an apparatus of the invention can contain in-line solenoid valves 98 and 100 between the synthesis plate vacuum chuck and the waste container. These normally closed solenoids can be activated by the main vacuum system and act to isolate the waste container from the synthesis filter plate after the plate is evacuated.

The reagent containers are capable of holding liquid reagents. In one embodiment, the reagent containers are moisture-resistant. Moisture-resistant containers do not allow moisture from the outside environment to penetrate to the inside of the container. A moisture-resistant container is made of or coated with a moisture-resistant material such as stainless steel, glass or a plastic. Reagent containers are also resistant to the material that they hold, for example, a reagent container that holds a solvent such as acetonitrile is a solvent-resistant container.

To assure that the reagents being used are moisture free, the reagent containers, for example, glass bottles, can be cleaned and oven dried before the reagents are mixed. The bottles are filled within a dry box and molecular sieves are added and allowed to settle for 24 hours before the reagent is used. In addition, a Teflon filter is added to the intake line that is inserted into the reagent containers. This decreases the amount of fines or other small particles from sieves introduced into the intake lines and introduced into the synthesis plate.

Additional components of the synthesizer that can be stored within cabinet 10 and made accessible through doors 26 and 28 are shown in FIG. 5. These components include computer 102, a controller box 104 that controls the solenoid valves and a kill switch 106 to turn off the comptroller. Other components include solenoid valves 108, 110, 112, 114, 116 and 118 for controlling vacuum inlet to organic waste containers and other gas regulation functions as described below.

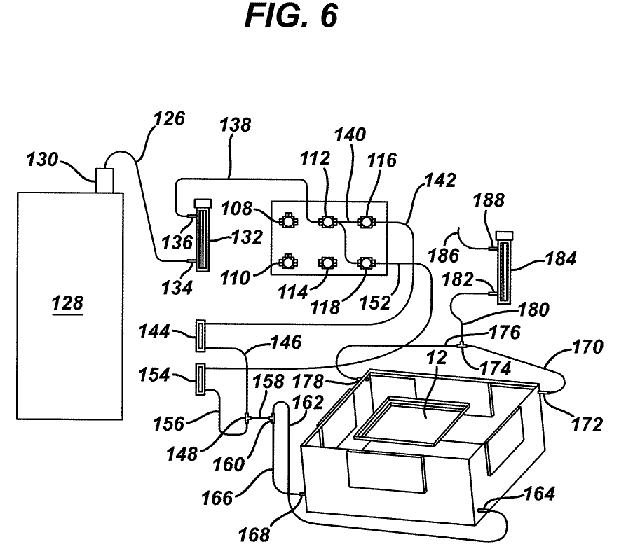

FIG. 6 shows a detailed diagram for gas flow in dry box 12. The boiloff from a liquid nitrogen Dewar is used for dry box gas purge. As seen in the diagram, tubing 126 connects a liquid nitrogen Dewar 128 and gas regulators 130 to a gas dryer 132 via a lower gas dryer inlet port 134. Tubing 136 connects an upper gas dryer outlet port 138 to a main gas control solenoid valve 112. As shown, tubing 140 connects main gas control 112 to high flow control solenoid valve 116 and tubing 142 connects high flow control solenoid valve 116 to a high flow meter 144. Further, tubing 146 connects high flow meter 144 to a three-way connector 148. Tubing 150 connects main gas control solenoid valve 112 to a low flow control solenoid valve 118 and tubing 152 connects low flow control solenoid valve 118 to a low flow meter 154 and tubing 156 connects low flow meter 154 to three-way connector 148. Tubing 158 connects three-way connector 148 to a three-way connector 160. Tubing 162 connects three-way connector 160 to a gas inlet port 164 and tubing 166 connects three-way connector 160 to a gas inlet port 168. Tubing 170 connects a gas outlet port 172 to a three-way connector 174 and tubing 176 connects gas outlet port 178 to three-way connector 174. Gas inlet and outlet ports 164, 168, 172 and 178 are connected to dry box 12. Tubing 180 connects three-way connector 174 to a gas inlet port 182 on a gas dryer 184 and tubing 186 connects a gas outlet port 188 on gas dryer 184 to the atmosphere for venting.

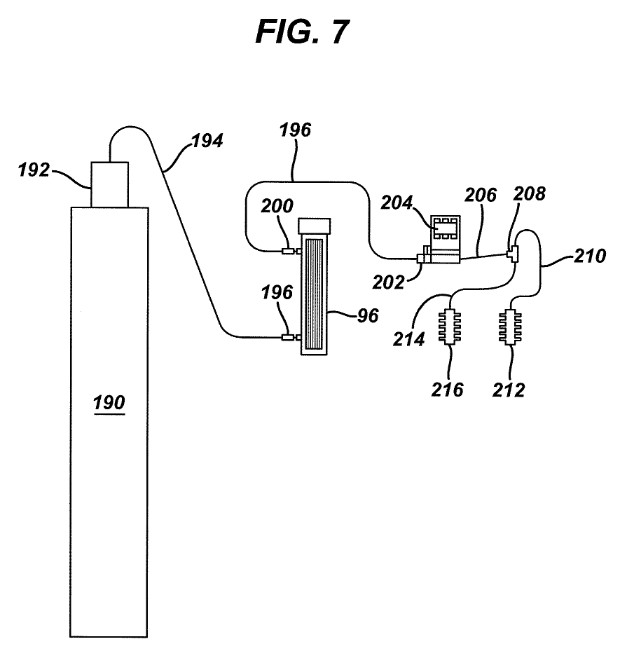

FIG. 7 shows a detailed diagram of a reagent bottle pressure system. The diagram shows a regulated helium gas supply 190 and gas regulator 192, tubing 194 connecting regulated helium gas supply 190 and gas regulator 192 and a gas inlet port 196 on gas dryer 96. Tubing 198 connects a gas outlet port 200 on gas dryer 96 with an inlet port 202 on a digital gas regulator 204. Tubing 206 connects digital gas regulator 204 with a three-way connector 208. Tubing 210 connects three-way connector 208 with a gas supply manifold 212 and tubing 214 connects three-way connector 208 with a gas supply manifold 216. The gas supply manifolds feed each reagent container 72-92 (not shown on FIG. 5).

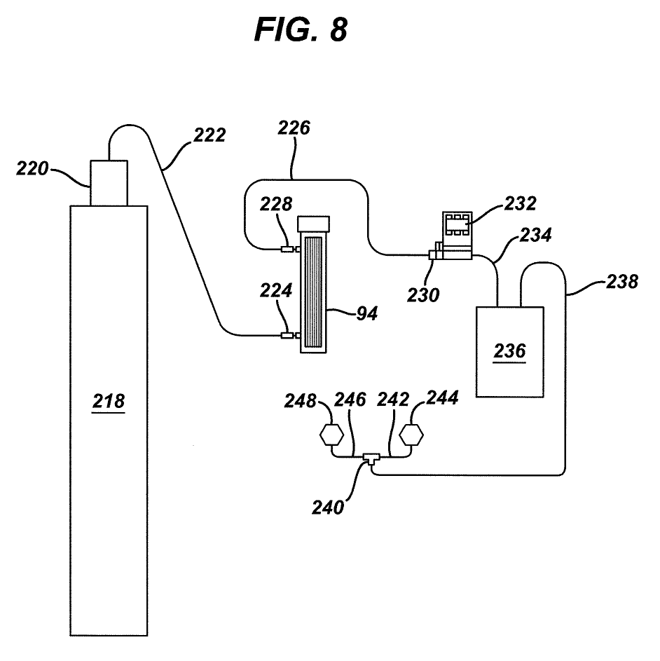

FIG. 8 shows a detailed diagram of an acetonitrile (ACN) wash system. The diagram shows a regulated helium gas supply 218 and gas regulator 220, tubing 222 connecting regulated helium gas supply 218 and gas regulator 220 and a gas inlet port 224 on a gas dryer 94. Tubing 226 connects a gas outlet port 228 on gas dryer 94 with an inlet port 230 on a digital gas regulator 232. Tubing 234 connects digital gas regulator 232 with an acetonitrile dewar 236. Tubing 238 connects acetonitrile dewar 236 with a three-way connector 240. Tubing 242 connects three-way connector 240 with a solenoid valve wash line manifold 244 and tubing 246 connects three-way connector 240 with a solenoid valve wash line manifold 248. In one embodiment of the invention, two further solenoid valve wash line manifold are provided so that a total of 24 wash lines are fed.

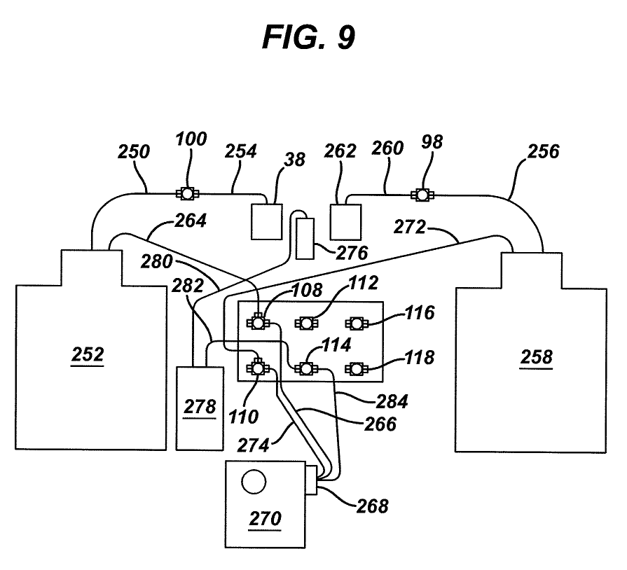

FIG. 9 shows a detailed diagram of a vacuum system. The diagram shows tubing 250 connecting a waste container 252 with solenoid valve 100 and tubing 254 connecting solenoid valve 100 with synthesis plate (plate 1) 38. The diagram also shows tubing 256 connecting a waste container 258 with solenoid valve 98 and tubing 260 connecting solenoid valve 98 with a synthesis plate (plate 2) 262. Tubing 264 connects waste container 252 with a three-way vacuum inlet solenoid valve 108 and tubing 266 connects three-way vacuum inlet solenoid valve 108 with a dry Teflon vacuum pump 268 and trap 270. Likewise, tubing 272 connects waste container 258 with three-way vacuum inlet solenoid valve 110 and tubing 274 connects three-way vacuum inlet solenoid valve 110 with dry Teflon vacuum pump 268 and trap 270. Drain 276 is connected to a drain waste container 278 with tubing 280 and tubing 282 connects a drain waste container 278 with vacuum inlet solenoid valve 114. Tubing 284 connects vacuum inlet solenoid valve 114 with dry Teflon vacuum pump 268 and trap 270. In a preferred embodiment, similar connections, tubing and valves are used to accommodate two additional plates, so that the system is configured for a total of four plates.

As discussed above, conventional automated polynucleotide synthesizers have the ability to produce varied polynucleotides in parallel. However, the prior art systems all require that the synthesis reaction occur synchronously in each of the reaction wells. For example, although the bases may differ, the 20^{th} base is added to each polynucleotide at the same time. Correspondingly, the automated run must continue until the longest polynucleotide is synthesized even though shorter polynucleotides may have been completed earlier. Similarly, each reaction well undergoes capping, deoxidation, deprotection and washing at the same time as well. Thus, during reactions having significant wait times, the entire system is idle.

The present invention increases the flexibility and performance of automated synthesis by performing reactions in at least two different reaction volumes asynchronously. Specifically, during a wait time for a reaction in one well, the injection head is used to deliver a reagent to another well, so that the synthesis cycle in that well can continue without waiting for the reaction to complete in the first well. For example, during the wait time for the coupling step in a first plate, a deblocking, capping or oxidation step can be performed in a second plate. Further, performing a reaction asynchronously also includes conducting a coupling step on a second plate during the reaction wait time corresponding to the coupling of a base in a different position on the polynucleotide chain. For example, coupling of the 10^{th} base to the polynucleotides in the second plate can be initiated while the reaction coupling the 9^{th} base to the polynucleotides in the first plate is occurring.

By using all available wait times to perform additional reaction steps in alternate plates, the amount of idle time for the system can be reduced dramatically.

In a further embodiment of the invention, more than one additional reaction step is performed on one or more additional plates during the reaction wait time of the first plate.

In the embodiment of the invention described above, four separate 96-well plates are employed. By grouping polynucleotides of similar length to be synthesized on the same plate, each plate can be removed as soon as the longest polynucleotide on the plate is synthesized. This advantage is realized in any embodiment using more than one plate. The combination of air lock 18 and gloved access points 14 and 16 facilitate the removal of one plate from the synthesis platform without contaminating the atmosphere, allowing synthesis to continue on the remaining plates. Thus, in a preferred embodiment, two separate plates are used. More preferably, three separate plates are used. Even more preferably, four separate plates are used. As one having skill in the art will appreciate, the concepts of the present invention can also be extended to embodiments having five or more separate plates.

As discussed above, the phosphoramidite polynucleotide synthesis cycle generally comprises four steps, described in detail below: (1) Deblocking; (2) Activation/coupling; (3) Capping; and (4) Oxidation.

Deblocking: The synthesis cycle begins with the removal of the DMT group from the 5′ hydroxyl of the 5′-terminal base by brief exposure to dichloroacetic acid (DCA) or trichloroacetic acid (TCA) in dichloromethane (DCM). The yield of the resulting trityl cation can be measured to help monitor the efficiency of the synthetic reaction. Protection of the reactive species (primary amines and free hydroxyls), on the nucleoside building blocks insures that the exposed 5′-hydroxyl is the only reactive nucleophile capable of participating in the coupling reaction (next step).

Activation/Coupling: DNA phosphoramidites are converted to a more reactive form by treatment in tetrazole or a tetrazole derivative at the time of coupling. These processes occur through the rapid deprotonation of the phosphoramidite followed by the reversible and relatively slow formation of a phosphorotetrazolide intermediate. Coupling reactions with activated deoxyribonucleoside-phosphoramidite reagents are fast and efficient. An excess of tetrazole over the phosphoramidite can be used to ensure complete activation and an excess of phosphoramidite over reactive polynucleotide coupled to CPG. Under these types of conditions coupling efficiencies of >99% can be achieved.

Capping: Since the base-coupling reaction is not 100% efficient, a small percentage of the growing polynucleotides on CPG supports will fail to couple and result in undesired, truncated species. Unless blocked, these truncated or partial polynucleotide products can continue to function as a substrate in later cycles, extend, and result in near full-length polynucleotides with internal deletions. These truncated or partial polynucleotide products are called (n−1)-mer species. These “reaction failures” can be mostly prevented from participating in subsequent synthesis cycles by “capping”, which involves acetylation of the free 5′-OH with acetic anhydride. In a preferred embodiment, the CapA reagent comprises tetrahydrofuran acetic anhydride and the CapB reagent comprises tetrahydrofuran pyridine-N-methylimidazole.

Oxidation: At this point, the DNA bases are connected by a potentially unstable trivalent phosphite triester. This species is converted to the stable pentavalent phosphotriester linkage by oxidation. Treatment of the reaction product with dilute iodine in water/pyridine/tetrahydrofuran forms an iodine-phosphorous adduct that is hydrolyzed to yield pentavalent phosphorous. The oxidation step completes one cycle of polynucleotide synthesis; subsequent cycles begin with the removal of the 5′-DMT from the newly added 5′-base. Alternatively, a subsequent capping step can follow the oxidation step.

After synthesis is complete, cleavage and deprotection reactions finalize production of the polynucleotide. The polynucleotide is cleaved from the solid support with concentrated ammonium hydroxide at room temperature. Continued incubation in ammonia at elevated temperature deprotects the phosphorus via S-elimination of the cyanoethyl group and also removes the protecting groups from the heterocyclic bases.

In a presently preferred embodiment of the invention, an automated polynucleotide synthesizer, such as synthesizer 10, is used to deliver reagents to a plurality of 96-well plates. First, a pre wash with acetonitrile is performed with a pair of injector pins from group 70, to deliver a total volume of 500 μL. This step takes 6 seconds to perform per plate. Next, a precapping step is performed with the CapA and B reagents from injector pins in groups 64 and 66. Two injections are performed to deliver a final volume of 220 μL. This step takes 16 seconds to perform per plate and requires a wait time of 75 seconds. Before the reiterative cycle begins, there is a further acetonitrile wash with a pair of injector pins from group 70, to deliver a total volume of 500 μL. This step takes 6 seconds to perform per plate.

The next five steps comprise the core synthesis cycle, wherein a nucleotide base is annealed to the growing molecule chain with each iteration. Each step is followed by a wash. First, a deblocking reaction with DCA is initiated by injecting two volumes with injector pins from group 62 to deliver a final volume of 100 μL. This step requires 6 seconds to perform per plate and requires a wait time of 50 seconds. The acetonitrile wash is performed with a pair of injector pins from group 70, to deliver a total volume of 500 μL. This step takes 6 seconds to perform per plate. Second, the coupling reaction is initiated by injecting 100 μL of an amidite from one of the injector pins 48-56 together with 100 μL of the activator reagent, for a total injection volume of 200 μL. This step requires 40 seconds per plate and requires a wait time of 250 seconds. The acetonitrile wash is performed with one injector pin from group 70, to deliver a total volume of 250 μL. This step takes 6 seconds to perform per plate. Third, the capping reaction is performed with the CapA and B reagents from injector pins in groups 64 and 66. One injection is performed to deliver a final volume of 110 μL. This step takes 16 seconds to perform per plate and requires a wait time of 75 seconds. The acetonitrile wash is performed with one injector pin from group 70, to deliver a total volume of 250 μL. This step takes 6 seconds to perform per plate. Fourth, the oxidation reaction is performed by injecting iodine with an injector pin from group 68. One injection is performed to deliver a final volume of 50 μL. This step takes 6 seconds to perform per plate and requires a wait time of 70 seconds. Fifth, another capping reaction is performed with the CapA and B reagents from injector pins in groups 64 and 66. One injection is performed to deliver a final volume of 110 μL. This step takes 16 seconds to perform per plate and requires a wait time of 75 seconds. The acetonitrile wash is performed with one injector pin from group 70, to deliver a total volume of 250 μL. This step takes 6 seconds to perform per plate.

After the last nucleotide base is added to the polynucleotide chain, a final deblocking step is performed with three DCA injections from pins in group 62 to deliver a final volume of 300 μL. A final acetonitrile wash is performed with three injections from pins in group 70, to deliver a total volume of 750 μL.

As can be seen, significant wait times are required following the deblocking, coupling, capping and oxidations steps. Specifically, deblocking requires 50 seconds, coupling requires 250 seconds, capping requires 75 seconds and oxidizing requires 70 seconds. Further, all times required to deliver the reagents to each well in a plate is less than the shortest wait time. Specifically, the wash steps require only 6 seconds, the deblocking step requires 6 seconds, the coupling step requires 40 seconds, the capping step requires 16 seconds and the oxidizing step requires 6 seconds. Accordingly, regardless of which reaction is occurring in a first plate, there is sufficient time to perform at least one reaction step in at least one additional plate during the wait time. Preferably, a plurality of additional reaction steps are performed on one or more additional plates during the wait time.

In another embodiment of the invention, injection volumes can be increased to give the following results. Prewash with total volume of 1200 μL, precap with total volume of 440 μL, wash with total volume of 1200 μL, deblock with total volume of 200 μL, wash with total volume of 1200 μL, couple with total volume of 400 μL, wash with total volume of 600 μL, cap with total volume of 220 μL, wash with total volume of 600 μL, oxidize with total volume of 80 μL, wash with total volume of 600 μL, cap with total volume of 220 μL, wash with total volume of 600 μL, final deblock with total volume of 300 μL, and final wash with total volume of 1800 μL.

In yet another embodiment of the invention, exemplary injection volumes are as follows, for nucleotide base, activator, cap B and deblock, each 100 μL, wash is 650 μL, oxidizer is 80 μL and cap A is 120 μL. In this embodiment, the following exemplary wait times can be used, 50 seconds for the deblock step, 270 seconds for the coupling steps, 100 seconds for the capping step and 70 seconds for the oxidizing step. A purge time of 18 seconds and a drain time of 2 seconds can also be used together with a vacuum time of 15 seconds for draining and 2 seconds for equalizing.

Computer 102 is preferably programmed to instruct controller 104 to exploit the reaction wait times together with the delivery times to allow the synthesis reactions to proceed in at least one reaction well during the wait time of a reaction occurring in another reaction well. Accordingly, a synthesizer embodying features of the invention can be controlled by a Windows® XP or Windows® 2000 computer. In one embodiment, the software is written in VisualBasic 6.0. Also preferably, the computer runs Compumotor Com control and Sigma Scan image analysis software to control the solenoid valves and xy table and monitor performance of the synthesis, respectively. In general, the software controls the machine operation.

In one embodiment, a startup procedure is performed by following a series of dialog boxes that prompt the operator through the necessary steps. Once the machine has been set up for a run and the synthesis procedure has been started no further user intervention is required. The software handles the table motion and valve operations, provides a continuous update on the status of the synthesis process, and performs the required shutdown steps once the synthesis is complete. In addition, there are preferably a series of options to allow the user to perform a variety of service and maintenance procedures (such as calibration of injection volumes and resetting plate offsets and well positions).

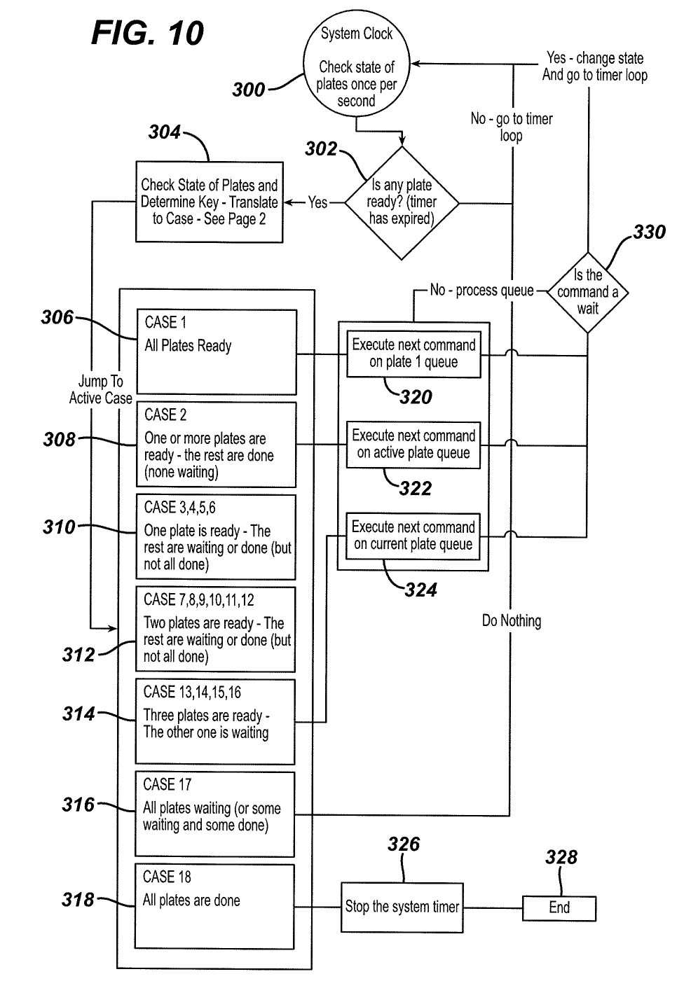

FIG. 10 shows a flowchart of a sequence of steps to govern the sequence of synthesis reaction steps performed on separate plates. In one embodiment of the invention, computer 102 is programmed to perform the steps shown in FIG. 10 with respect to four plates. In general, step 300 is driven by the system clock to check the state of each plate. Step 302 determines whether the wait timer for any plate has expired, thus indicating that at least one plate is ready for an additional reaction step. If no plate is ready, return to step 300 to allow another clock cycle to pass and recheck the state of each plate. If at least one plate is ready, step 304 translates the state of the plates to a code indicating one of 18 possible cases. Steps 306-318 correspond to the case determined in step 304, and the process performs the respective step as follows.

Step 306 applies if all plates are ready and this leads to the execution of step 320, which executes the next command on the plate 1 queue.

Step 308 applies if one or more plates are ready and the rest are done, with no plates waiting. This step leads to execution of step 322, which executes the next command on the active plate queue.

Step 310 applies if one plate is ready and the rest done or waiting, but not all done. This step leads to step 324, which causes the execution of the next command on the current plate queue. The active plate queue is the list of commands for the plate currently being serviced. There is only one plate active at a time. The current plate queue is the list of commands for the next plate that is ready to be serviced. When the system accesses that plate queue, it becomes the active plate queue or current plate queue.

Step 312 applies if two plates are ready, one plate is done and one plate is waiting. This step also leads to step 324, discussed above.

Step 314 applies if three plates are ready and one plate is waiting. This step also leads to step 324, discussed above.

Step 316 applies if all plates are waiting, or if some plates are waiting and some plates are done. This step returns the process to step 300 and the timer loop.

Finally, step 318 applies if all plates are done. If so, then step 326 stops the system timer and step 328 ends the process.

Following execution of step 320, 322 or 324, it is determined if the next command is a wait in step 330. If not, the process returns to the process queue of steps 320, 322 and 324. If the command is a wait, the state of the plates is updated and the process is returned to step 300 and the timer loop.

In one embodiment of the invention, the process for determining the state of the plates comprises assigning a four digit code. The thousands position corresponds to plate 1, the hundreds position corresponds to plate 2, the tens position corresponds to plate 3 and the ones position corresponds to plate 4. In each position, the digit 1 indicates that a plate is ready, the digit 2 indicates that a plate is waiting and the digit 3 indicates that a plate is done.

By implementing this code, the following 18 possible states are used to indicate the state of the plates. In case 1, the code has a value of 1111 indicating that all plates are ready.

In case 2, the code can have the values 1113, 1131, 1311, 3111, 1133, 1313, 1331, 3311, 3113, 3131, 1333, 3133, 3313, and 3331 indicating that at least one plate is ready and the rest are waiting. In case 3, the code can have the values 1223,1232, 1322, 1323, 1332, 1233 and 1222 indicating that plate 1 is ready, at least one plate is waiting and the rest are done. In case 4, the code can have the values 2123, 2132, 3122, 3123, 3132, 2133 and 2122 indicating that plate 2 is ready, at least one plate is waiting and the rest are done. In case 5, the code can have the values 2213, 2312, 3212, 3213, 3312, 2313 and 2212 indicating that plate 3 is ready, at least one plate is waiting and the rest are done. In case 6, the code can have the values 2231, 2321, 3221, 3231, 3321, 2331 and 2221 indicating that plate 4 is ready, at least one plate is waiting and the rest are done.

In case 7, the code can have the values 1123, 1132 and 1122 indicating that plates 1 and 2 are ready, at least one plate is waiting and the rest are done. In case 8, the code can have the values 1213, 1312 and 1212 indicating that plates 1 and 3 are ready, at least one plate is waiting and the rest are done. In case 9, the code can have the values 1231, 1321 and 1221 indicating that plates 1 and 4 are ready, at least one plate is waiting and the rest are done. In case 10, the code can have the values 2113, 3112 and 2112 indicating that plates 2 and 3 are ready, at least one plate is waiting and the rest are done. In case 11, the code can have the values 2131, 3121 and 2121 indicating that plates 2 and 4 are ready, at least one plate is waiting and the rest are done. In case 12, the code can have the values 2311, 3211 and 2211 indicating that plates 3 and 4 are ready, at least one plate is waiting and the rest are done.

In case 13, the code can have the value 2111 indicating that plates 2, 3 and 4 are ready and plate 1 is waiting. In case 14, the code can have the value 1211 indicating that plates 1, 3 and 4 are ready and plate 2 is waiting. In case 15, the code can have the value 1121 indicating that plates 1, 2 and 4 are ready and plate 3 is waiting. In case 16, the code can have the value 1112 indicating that plates 1, 2 and 3 are ready and plate 4 is waiting.

In case 17, the code can have the values 2223, 2232, 2322, 3222, 2233, 2332, 3322, 2323, 3232, 2333, 3233, 3323 and 3332 indicating that at least one plate is waiting and the remaining plates are done.

Finally, in case 18, the code has the value 3333 indicating that all plates are done. As indicated above, each case corresponds to one of steps 306-318.

An apparatus of the invention also regulates the synthesis environment to optimize conditions for highly efficient synthesis. Polynucleotide chemistry is known to be particularly sensitive to the presence of water vapor and air (Gait “Oligonucleotide Synthesis: A practical-approach” Oxford University Press, New York, N.Y., 1984). The efficiency of the coupling reactions is significantly reduced by moisture. To address this sensitivity, an apparatus of the invention preferably maintains a closed continuous anhydrous system for automated polynucleotide synthesis. An advantage of such an apparatus is that humidity is decreased during the polynucleotide synthesis reactions. A reduction in humidity or moisture within the automated polynucleotide synthesis system results in increased coupling efficiency. Increased coupling efficiency results in greater yields at each step and the ability to synthesize longer polynucleotides. Increased coupling efficiency also reduces the amount of partial polynucleotide products, increasing the quality of the final polynucleotide product.

Another advantage of an apparatus of the invention is that the apparatus can regulate pressure stability. Stable pressure can reduce variation in the delivery of chemicals in the synthesis reaction. For example, as the synthesis reaction continues, there is a drop in reagent container volume and gas pressure in high pressure gas cylinders of high purity gas which results in a concomitant drop in pressure. This drop in pressure can result in a change in the amount of reagent that is delivered in the synthesis reaction that can reduce coupling efficiency. Components of an apparatus of the invention, such as the digital gas regulator, can monitor gas pressure in real time and can react resulting in the equalization of pressure to a more constant level. Maintaining constant pressure results in more consistent delivery of the correct amount of reagent in the synthesis reaction which results in better coupling efficiency. Maintaining constant pressure in the system also aids in reducing relative humidity in the system.

An apparatus of the invention can regulate or control a homeostatic state, for example, with low moisture content and a steady pressure level for the consistent delivery of chemical reagents. Both the decrease in humidity and decrease in variation in chemical delivery can result in higher coupling efficiency that allows for the production of polynucleotides of longer length and higher quality. Polynucleotides of high quality can be used without a purification step. Purification steps are time consuming, labor intensive, and result in lower yield of the final product. Polynucleotides of long length or high quality are useful in several applications including, for example, gene assembly and site-directed mutagenesis.

Polynucleotide synthesis efficiency is typically about 98-99% for each cycle of chemistry, so for each cycle about 1-2% of the reaction products will be 1 base shorter than expected. Some truncated species fail “capping” and continue to participate in additional cycles of DNA synthesis. For a 60-mer polynucleotide, less than 50% of the final product will be the desired full-length molecules. The final synthesis product will include a mixed population of (n−1)-mer and (n−2)-mer (etc.) molecules which represent a heterogeneous collection of sequences, effectively a pool of deletion mutants at every possible position.

Synthesis scale refers to the amount of starting material while synthesis yield refers to the amount of final product recovered after the synthesis and purification steps have been completed. In polynucleotide synthesis, the 3′ terminal base is attached to a solid support at the scale ordered by the customer. Bases are added one at a time in the 3′ to 5′ direction. Ideally, each added base would couple with 100% efficiency, resulting in 100% yields. In reality, coupling efficiency is somewhat less than 100%, and this small decrease can result in a substantial decrease in yield of the final oligonucleotide (since the effects of coupling efficiency will be additive). Moreover, coupling efficiency can vary for each base added, therefore the sequence itself can contribute to wide variations in yields. For a 250-nmole-scale reaction, the final yield after deprotection and purification can range from 10 to 100 nmoles. Some sequences tend to produce higher yields than others, and this trend is usually reproducible. The yield for the synthesis of one 20-base sequence can be twice that obtained for a different 20-base sequence, even if the two sequences are run on the same day, on the same machine, using the same reagents. Some variability in yields can also be derived from the individual machine used.

Theoretical yield for a given synthesis is (E_{ff})ⁿ⁻¹ with “E_{ff}” representing coupling efficiency and “n” representing the number of bases in the polynucleotide. If the coupling efficiency is 99% (E_{ff}=0.99), the fraction of full-length product present after synthesis will be approximately (0.99)¹⁹ or 83% for a 20-mer; (0.99)⁴⁹ or 61% for a 50-mer; and (0.99)⁷⁴ or 48% for a 75-mer. A small decrease in coupling efficiency will result in a substantial decrease in expected yield. For example, if coupling efficiency is 99%, the yield for a 100-mer is (0.99)⁹⁹ or 37%, but if the coupling efficiency drops to 98%, yield falls to (0.98)⁹⁹ or 13%.

However, coupling efficiency varies with each base added. Coupling efficiency is lower for the first five to six bases, presumably because of steric hindrance near the surface of the solid support. Coupling efficiency then increases to an optimum of about 99%, as is characteristic for the addition of the twentieth base, and then once again, falls to suboptimal levels as length increases. Since coupling efficiency actually decreases as the polynucleotide becomes very long, yields on 100-mers can often be less than 10%. Product is also lost during any purification process, if done, which further decreases yields.

As described above, the efficiency of polynucleotide synthesis is maximized by providing an anhydrous reaction environment. Thus, in preferred embodiments of the invention, one or more features designed to restrict the entrance of moisture into the system. Accordingly, an apparatus of the invention can comprise a closed continuous anhydrous system for automated polynucleotide synthesis, including, for example, moisture-resistant reagent containers, a sealed dry box enclosing the synthesis platform, gloved access points, an airlock, an integrated desiccant chamber for storing reagents, moisture-resistant tubing connections, and in line gas dryers.

The apparatus of the invention contains several seals, for example, a seal between the dry box and synthesis platform and seals between connectors and containers of reagents. A seal is intended to mean a closure forming an airtight connection. A seal can be made of any material capable of making an airtight connection, for example, with glass, plastic or metal. Additionally, seals can be made of solvent-resistant material. Sealing materials include, for example, rubber, TYGON, or silicone. In one embodiment, connections in the apparatus of the invention are sealed to exclude moisture entry. In another embodiment, the connections are sealed with silicone caulk. In a further embodiment, the connection are double-sealed.

Seals used in the apparatus should be of sufficient strength to maintain an airtight connection. The strength of a seal can be measured, for example, by its ability to maintain a vacuum or pressure of a certain strength. The sealed connection of an apparatus of the invention can maintain a pressure of greater than 100 psi, greater than 75 psi, greater than 50 psi, or greater than 25 psi. In one embodiment, the sealed connections of an apparatus of the invention can maintain a pressure of greater than 25 psi.

The seams of dry box 12 and the associated components can be double sealed, inside and out, and the gaskets on the access doors are reinforced with silicon sealer. In addition, the cable couplings are sealed and the open tube couplings that are used to feed the injection lines through are sealed using silicone rubber sealant.

An apparatus of the invention preferably contains a humidity meter inside the dry box. For example, the humidity meter can be a digital humidity meter. The meter can allow the internal dry box humidity to be continually monitored by the system in real time. This data can then be fed into a computer, manually or automatically, and used to determine when synthesis should begin as opposed to waiting a predetermined period of time before beginning synthesis. For example, synthesis can be programmed to begin when the humidity within the dry box is less than or equal to 1% humidity. In addition, the system can alert the operator if the humidity is above a specific amount and suspend the synthesis reaction if necessary.

The efficiency of the synthesis reaction can be improved by the addition of a humidity meter since the reaction can not begin or can not proceed if the humidity content of the synthesis dry box is too high. In addition, beginning the synthesis reaction based on the humidity level instead of a set period of time can speed up the synthesis reaction if the time needed to reduce the humidity to an acceptable level is less than the set period of time. Humidity meters are commercially available, for example, from Dickinson such as the Dickinson Model TP120 SN 02221347.

In the embodiment of the invention described above with regard to FIG. 7, flow through gas dryers 94 and 96, for example, are connected to tubing that connects a reagent container 72-92 to a gas supply, also known as reagent gas feeds. The reagent gas feed is made of material that can withstand the desired pressure level. For example, a reagent gas feed can be plastic, stainless steel, or Teflon tubing that connects a gas cylinder with a wash solution container. Various types of tubing can be used for the reagent gas feed, for example, the tubing can have different levels of flexibility or different diameters so long as the tubing is capable of carrying a gas from a gas source to a reagent container. As also described above, a flow through gas dryer 132 can be connected between a gas supply and the dry box 12. In a further embodiment, an apparatus of the invention contains a flow through gas dryer connected to the dry box gas outlet port, a flow through gas dryer connected to the reagent gas feed used to pressurize nucleotide solution containers, and a flow through gas dryer connected to the reagent gas feed used to pressurize the acetonitrile wash solution container (also known as a Dewar).

In one embodiment, the reagent gas feed is made of moisture- and solvent-resistant tubing. Because a gas feed carries pressurized gas, tubing and seals of appropriate strength and composition are used. For example, the gas feeds can be Teflon tubing that is connected at one end to a gas cylinder using Swagelok® pressure pipe fitting and at the other end to a reagent container or containers using Swagelok® pressure pipe fittings. A reagent gas feed can connect a reagent such as a nucleotide solution to a gas cylinder, for example, a helium gas cylinder. Helium can be used to pressurize the reagent bottles that can prevent bubbles in the delivery lines. In addition, a reagent gas feed can connect a reagent such as a wash solution, for example, acetonitrile, to a gas cylinder as diagrammed in FIG. 8.

An apparatus of the invention can contain at least one in-line solenoid valve 98 and 100 between the synthesis plate vacuum chuck and the waste container. These normally closed solenoids can be activated by the main vacuum system and act to isolate the waste container from the synthesis filter plate after the plate is evacuated as shown in FIG. 9. This can prevent the waste container from equalizing and allow the container to be kept under continuous negative pressure (vacuum). The resulting effect is that the system can vacuum out the synthesis plate immediately, rather than first needing to pump down the waste container. In another embodiment, an apparatus of the invention can have three way vacuum inlet solenoid valves 108 and 110 that are re-routed and shunted to prevent equalization (see FIG. 9). A high strength vacuum system, such as a Welch self-cleaning Teflon dry Vacuum System Model 2025 (Gardner Denver Thomas, Inc., Skokie, Ill.) can be used in an apparatus of the invention.

Several types of gases can be used in an apparatus of the invention. The gases used in an apparatus of the invention can be stable or inert gases that contain little reactivity on their own. For example, noble gases such as helium, neon, argon, krypton, xenon, and radon are inert gases that can be used in the apparatus. In addition, a gas such as nitrogen can be used in an apparatus of the invention as an inert gas. In one embodiment, the gas used in an apparatus of the invention is nitrogen, argon, or helium. In another embodiment, helium is used to pressurize reagent containers and nitrogen is used in the dry box. The nitrogen gas can be derived, for example, from a liquid nitrogen (N₂) boil off Dewar. An advantage to using nitrogen is that it is an inexpensive gas.

The tubing can be, for example, moisture- and solvent-resistant tubing. Several types of moisture- and solvent-resistant tubing are known in the art and commercially available, such as plastic and TYGON®, Teflon®, and polypropylene tubing. Preferably, the tubing used in the apparatus is Teflon® tubing.

Tubing can be connected to reagent containers in a variety of ways. For example, tubing can be removably connected to the other components of the apparatus such as the reagent containers. This allows for rapid and convenient adjustment or replacement of the tubing and changing of the containers. In one embodiment a removable connection can be achieved by stretching the tubing over the outer surface of opening such as an inlet or outlet port of the container. The tubing stretched over the outer surface of the inlet or outlet port can be held in place by a clasp such as an elastic ring or a metal clasp. Also, for example, the tubing can be held in place by an outer sheath that wraps around an outer surface of an opening such as an inlet or outlet port on one end and an outer surface of an inlet or outlet port on the other end to form an airtight closure. A convenient outer sheath can be a short section of tubing including, for example, TYGON® tubing. Also, for example, Swagelok® Parker pressure pipe fittings, polypropylene and stainless steel fittings can be used to connect tubing to various containers.

As shown in FIGS. 7 and 8, an apparatus of the invention contains a digital gas regulator 204 and 232 where the gas regulator maintains constant pressure in the reagent containers. In one embodiment, the gas regulator is a digital gas regulator. A gas regulator can monitor the level of gas pressure accurately in real time and is capable of making adjustments to the level of gas in order to keep gas pressure constant. A constant level of gas pressure is a level of pressure that may fluctuate slightly around a desired value. For example, as the volume of a reagent drops, the pressure in the reagent container changes. This change is rapidly detected by the gas regulator and a signal is sent that results in regulation of the gas pressure back to the desired level. The gas regulator can quickly react to small changes in gas pressure such that the level of gas pressure is essentially constant, although small changes in gas pressure can be experienced for short periods of time. If for some reason the pressure in the system drops below a specific amount, the system can alert the operator and suspend the synthesis reaction if necessary. In this way a continuous homeostatic system is maintained. A digital signal allows for more accurate adjustment of gas pressure than the use of an analog signal. Hence a digital signal allows for contemporaneous adjustment of gas pressure. A digital gas regulator can be used to maintain gas pressure, for example, at increments of 0.1 psi pressure, 0.05 psi pressure, or 0.01 psi pressure. The more accurate the gas regulator, the more accurate the control of pressure within the system. Digital gas regulators are commercially available, for example, from Alicat Scientific (Tucson, Ariz.).

As used herein, the term “digital gas regulator” is intended to mean a device that monitors gas pressure accurately over time and is capable of sending an output signal to a device which functions to adjust gas pressure to a desired level. A digital gas regulator can be set to monitor gas pressure and maintain a constant level of gas pressure in a system. For example, a digital gas regulator can monitor the level of gas pressure in a pressurized reagent container, high pressure gas cylinder, or closed system such that, when the level of reagent in the container changes or pressure in the gas cylinder changes, the resulting change in pressure in the container is accurately monitored by the digital gas regulator and shown on a digital display. The digital gas regulator can then send a signal to a valve that controls the amount of gas that enters the reagent container adjusting the amount of gas entering the container to equalize the gas pressure in the container.

The flow through gas dryers connected to the dry box gas inlet and outlet ports can be used to help ensure that the gas being introduced from the liquid nitrogen dewar into the dry box is pre-dried and that little to no moisture is introduced via back flow from the dry box exhaust. The flow through gas dryers connected to the reagent gas feeds help to ensure that moisture is not introduced into the reagents or wash chemicals.

The flow through gas dryers are any drying device situated in line with a connector, such as tubing, so that the material in the connector can flow through the drying device. Thus, the drying device can be any device that removes moisture from the material in the connector. For example, the drying device can contain a desiccant material absorbs moisture from the material in the connector. In addition, a desiccant material can be put inside the dry box or in any location to help reduce moisture content. Many desiccants are known in the art and commercially available, for example, clay based substances and zeolites. Commercially available desiccants include, for example, DRIERITE® and Siliporite NK10F. In one embodiment, the desiccant material used in an apparatus of the invention is phosphorous pentoxide and sodium hydroxide. In another embodiment, the desiccant material used in an apparatus of the invention is DRIERITE® and 5A Molecular Sieve. A desiccant material such as DRIERITE® can dry gasses to a dryness of 0.005 mg/l of air.

A flow through gas dryer can be, for example, a DRIERITE® Gas Purifier which is filled with indicating DRIERITE® and 5A Molecular Sieves for the removal of moisture, impurities and particulates from gas lines. Such a gas purifier can be attached using compression type tube fittings. DRIERITE® changes color from blue to pink to indicate exhaustion of drying capacity. DRIERITE® can be replaced or regenerated by known procedures. The 5A Molecular Sieves remove impurities that have an effective molecular diameter of less than 5 angstroms. The DRIERITE® Gas Purifier is commercially available. It has a column made of molded polycarbonate and a polycarbonate cap fitted with an o-ring gasket. The DRIERITE® and molecular sieves are held in place between felt filters. The bed supports and coil springs are stainless steel and the outlet frit is 40 micron. The dimensions of the column are 2⅝ inches by 11⅜ inches. The connections are ⅛ inch stainless steel male tube fittings. The recommended maximum working pressure is 100 psig and water capacity is 25 grams. The recommended flow rate is up to 300 liter per hour for maximum efficiency.

As described further above, the connections in the above described apparatus can be sealed to exclude moisture entry, for example, using silicone caulk. Also the sealed connections can maintain a pressure of greater than 100 psi strength. Also as described above, the reagent containers can be nucleotide solution containers or waste solution containers, and in one embodiment, the reagent containers are a wash solution container and one or more nucleotide solution containers. The apparatus can further contain at least one flow through gas dryer connected to the tubing that pressurizes the reagent containers delivering dry reagents to the dry box, or connected to a reagent gas feed. Further, the apparatus can contain a humidity meter.

The invention provides an apparatus for maintaining a closed continuous system for automated polynucleotide synthesis. An apparatus of the invention can have several reagent containers; a dry box capable of forming a seal over a synthesis platform of an automated polynucleotide synthesizer; moisture-resistant tubing connecting the reagent containers to the dry box; a reagent gas feed connecting the reagent containers to a gas, and a digital gas regulator connected to the reagent gas feed. When tubing is in locations within the apparatus that are in contact with solvents, the moisture-resistant tubing chosen is also solvent-resistant. Several materials that are resistant to different solvents are known in the art.

In addition, argon or another inert gas is pumped continuously into the dry box 12 and the integrated desiccator cabinet. The constant flow of gas minimizes contamination of the phosphoramidite and tetrazole lines by vapors from the deblocking and oxidizing lines. In contrast to a prior art oligonucleotide synthesizer which uses a combination of two chambers to reduce water vapor and air from the reactions, the claimed apparatus contains one continuous anhydrous dry box reaction chamber.

Several polynucleotide synthesizer devices which are known in the art, both custom-made and commercially available, can be incorporated into an apparatus of the invention. In one embodiment, an apparatus of the invention contains a polynucleotide synthesizer such as described in Rayner et al. (Genome Research 8:741-747 (1998)), which can be attached to, or a component of, an apparatus of the invention.

In addition to the polynucleotide synthesizer described by Rayner et al. supra, there are other high-throughput polynucleotide synthesizers available for the rapid synthesis of multiple polynucleotides. For example, a polynucleotide synthesizer designed and built by the Human Genome Center at Lawrence Livermore National Lab uses a multichannel format (Sindelar and Jaklevic, Nucleic Acids Res. 23:982-987 (1995)). This system also uses phosphoramidite chemistry, but is limited to 12 polynucleotides each run. AMOS, the polynucleotide synthesizer designed and built at the Genome Center at Stanford University uses the same chemistry and synthesizes directly into a 96-well format on a reaction scale similar to the MerMade synthesizer (Lashkari et al., Proc. Natl. Acad. Sci. USA 92:7912-7915 (1995)).

A polynucleotide synthesizer can have width of about 55 inches, a depth of about 26 inches and a height of about 72 inches. The synthesis reagents can be stored in the desiccant chamber as described above, in standard pressurized media bottles and are transferred by Teflon lines to dry box 12 located at the top of cabinet 10. The electronics and computer controlling the machine within cabinet 10 as described, or can be located in a separate cart that can be placed either inside or outside the main frame as convenience dictates. Two argon tanks (to provide bottle pressure and an inert synthesis environment) can be strapped to the side of the frame.

Parameters necessary for the synthesis run can be stored in a group of simple text files that are accessed by the control software. These files contain the sequence for each polynucleotide as well as information about the injection volumes, the wait times for each stage in the synthesis cycle, the number of wash cycles after each stage, as well as the plate and well offsets and motor speed/acceleration for the xy table. These can be edited for each plate to allow different concentrations and yields for the plates. Further, as discussed above, by grouping polynucleotides of similar length on a single plate allows the plate to be removed as soon as synthesis of its longest polynucleotide is complete, while synthesis of longer polynucleotides continues uninterrupted on the remaining plates. Also, different polynucleotide sequences can be assigned to different plates to maximize the efficiencies represented by utilizing the wait time one a given plate to conduct asynchronous synthesis on other plates.

The reaction parameters can be adjusted to satisfactory operating requirements by evaluating polynucleotide quality using a combination of capillary electrophoresis (CE) high performance liquid chromatography (HPLC) and mass spectrometry. The CE and HPLC traces provide information about the % purity of N, whereas the HPLC traces can be used to quantify the amounts of residual chemicals left after the synthesis process is complete.

Described herein are presently preferred embodiments, however, one skilled in the art that pertains to the present invention will understand that there are equivalent alternative embodiments. As such, changes and modifications are properly, equitably, and intended to be, within the full range of equivalence of the following claims.

What is claimed is:

1. A method for the automated synthesis of polynucleotides comprising the steps of:
a) conducting a synthetic sequence comprising a repeating plurality of reactions in a first reaction volume, wherein each reaction is triggered by injecting reagents into the first reaction volume with an injection device and the reactions produce a polynucleotide; and
b) conducting a synthetic sequence comprising a repeating plurality of reactions in a second reaction volume, wherein each reaction is triggered by injecting reagents into the second reaction volume with an injection device and the reactions produce a polynucleotide;
wherein the synthetic sequence in the first reaction volume is asynchronous with the second synthetic sequence in the second reaction volume.

2. The method of claim 1, wherein the first reaction of the repeating plurality of reactions comprises adding a reagent to the first reaction volume and waiting a first period of time for the first reaction to occur, further comprising the steps of determining that sufficient time exists in the first period of time to conduct a second reaction in the second reaction volume; positioning the second reaction volume adjacent the injection device and injecting a reagent into the second reaction volume.

3. The method of claim 2, wherein the first reaction volume is located on a first plate and wherein the second reaction volume is located on a second plate.

4. The method of claim 3, further comprising the steps of completing a desired polynucleotide on the second plate; removing the second plate from a synthesis environment and continuing synthesis of another desired polynucleotide on the first plate.

5. The method of claim 4, wherein the step of removing the second plate from a synthesis environment comprises passing the second plate through an airlock while maintaining the synthesis environment.

6. The method of claim 4, wherein the step of removing the second plate from a synthesis environment comprises manipulating the second plate with at least one gloved access that maintains the synthesis environment.

7. The method of claim 1, further comprising the step of conducting the synthetic sequence in a third reaction volume, wherein the plurality of reactions are triggered by injecting reagents into the third reaction volume with the injection device; wherein the synthetic sequence in the first reaction volume is asynchronous with the synthetic sequence in the third reaction volume.

8. The method of claim 1, wherein the synthetic sequence in the second reaction volume is asynchronous with the synthetic sequence in the third reaction volume.

9. The method of claim 2, further comprising the steps of conducting the synthetic sequence in a third reaction volume, wherein the plurality of reactions are triggered by injecting reagents into the third reaction volume with the injection device, determining that sufficient time exists in the first period of time to conduct a third reaction in the third reaction volume; positioning the third reaction volume adjacent to the injection device and injecting a reagent into the third reaction volume.

10. The method of claim 7, further comprising the step of conducting the synthetic sequence in a fourth reaction volume, wherein the plurality of reactions are triggered by injecting reagents into the fourth reaction volume with the injection device; wherein the synthetic sequence in the first reaction volume is asynchronous with the synthetic sequence in the fourth reaction volume.

11. The method of claim 10, wherein the first, second, third and fourth reaction volumes are located on separate plates.

12. The method of claim 11, wherein the plates comprise 96-well plates.

13. The method of claim 1, wherein the first and second reaction volumes are located on separate plates, wherein the plates have a plurality of columns and a plurality of rows of reaction wells, further comprising the step of injecting a reagent to an entire column of the reaction wells simultaneously.

14. The method of claim 1, wherein the plurality of reactions comprise deblocking, coupling, capping and oxidizing.

15. The method of claim 1, further comprising the step of storing one or more reagents in an integrated desiccant chamber.

16. The method of claim 3, further comprising a fifth reaction volume located on the first plate, wherein the plurality of reactions are triggered by injecting reagents into the fifth reaction volume with the injection device and wherein the polynucleotide formed in the first reaction volume has a different sequence than the polynucleotide formed in the fifth reaction volume.

17. The method of claim 1, wherein the polynucleotide formed in the first reaction volume has a different molecular weight than the polynucleotide formed in the second reaction volume.

18. A system for the automated synthesis of polynucleotides, comprising a first reaction volume and a second reaction volume, an injection device having injectors for delivering reagents into the first reaction volume and the second reaction volume; an xy table configured to movably position the first reaction volume and the second reaction volume adjacent the injection device, and a controller configured to conduct a repeating plurality of reactions corresponding to a synthetic sequence in the first reaction volume and the second reaction volume, wherein the synthetic sequence in the first reaction volume is asynchronous with the synthetic sequence in the second reaction volume.

19. The system of claim 18, wherein the first reaction of the repeating plurality of reactions comprises adding a reagent to the first reaction volume and waiting a first period of time for the first reaction to occur and wherein the controller determines that sufficient time exists in the first period of time to conduct a second reaction in the second reaction volume, operates the xy table so that the injection device is positioned adjacent the second reaction volume and injects a reagent into the second reaction volume.

20. The system of claim 19, wherein the first reaction volume is located on a first plate and wherein the second reaction volume is located on a second plate.

21. The system of claim 20, further comprising a dry box for maintaining a reduced moisture atmosphere surrounding the first reaction volume, the second reaction volume, the injection device and the xy table.

22. The system of claim 21, wherein the dry box further comprises an air lock.

23. The system of claim 22, wherein the dry box further comprises at least one gloved access point.

24. The system of claim 21, further comprising an integrated desiccator chamber configured to store one or more reagents.

25. The system of claim 24, wherein the desiccator chamber and the dry box are configured so that one or more reagents are maintained under a reduced moisture atmosphere until delivery to the first and second reaction volumes.

26. The system of claim 18, further comprising a third reaction volume, wherein the xy table is configured to movably position the third reaction volume adjacent the injection device and wherein the controller is configured to conduct the repeating plurality of reactions in the third reaction volume such that the synthetic sequence in the first reaction volume is asynchronous with the synthetic sequence in the third reaction volume.

27. The system of claim 26, further comprising a fourth reaction volume, wherein the xy table is configured to movably position the fourth reaction volume adjacent the injection device and wherein the controller is configured to conduct the repeating plurality of reactions in the fourth reaction volume such that the synthetic sequence in the first reaction volume is asynchronous with the synthetic sequence in the fourth reaction volume.

28. The system of claim 27, wherein the first, second, third and fourth reaction volumes are located on separate plates.

29. The system of claim 28, wherein the plates comprise 96-well plates.

30. The system of claim 18, wherein the injection device is configured to deliver reagents to a plate comprising a plurality of rows and a plurality of columns of reaction wells and wherein the injection device is configured to deliver a reagent to an entire column of reaction wells simultaneously.

31. The system of claim 18, wherein the plurality of reactions comprise deblocking, coupling, capping and oxidizing.

32. The system of claim 19, wherein the controller includes software instructions comprising the steps of assessing the state of the first and second reaction volumes, determining the first reaction volume is waiting for a reaction to complete, determining the second reaction volume is ready for a subsequent reaction, and transmitting commands that cause the injection device to deliver to the second reaction volume to initiate the subsequent reaction.
